(19) United States
(12) Patent Application Publication
(10) Pub. No.: US 2026/0273007 A1
(43) Pub. Date: Sep. 17, 2026

(54) FEED ADDITIVE FOR IMPROVING PERFORMANCE OF FARMING ANIMALS

(71) Applicant: Nutreco IP Assets B.V., Boxmeer (NL)

(72) Inventors: David Manuel BRAVO, Boxmeer (NL); Karin BERGER BÜTER, Boxmeer (NL)

(21) Appl. No.: 19/162,369

(22) PCT Filed: Mar. 8, 2024
(86) PCT No.: PCT/EP2024/056249

§ 371 Date: Sep. 4, 2025

(30) Foreign Application Priority Data
Mar. 8, 2023 (EP) 23160803.5

(51) Int. Cl. A61K 36/484; A23K 10/30; A23K 50/10; A23K 50/75; A23K 50/80; A61P 3/00; A61P 31/04; A61P 31/20; A61P 33/02

(52) U.S. Cl. CPC A61K 36/484; A23K 10/30; A23K 50/10; A23K 50/75; A23K 50/80; A61P 3/00; A61P 31/04; A61P 31/20; A61P 33/02; A61K 2236/15; A61K 2236/331

(57) ABSTRACT

The present invention is in the field of feed additives for animals, e.g., farming animals, in particular feed additives to be added to animal feed to obtain an improved performance, e.g., an increasing feed intake, increasing average daily gain, increasing feed efficiency (i.e., decreasing feed conversion ratio), and/or increasing milk fat yield, of such animals.

### FIELD OF THE INVENTION

The present invention is in the field of feed additives for animals, e.g., farming animals, in particular feed additives to be added to animal feed to obtain an improved performance, e.g., an increasing feed intake, increasing average daily gain, increasing feed efficiency (i.e., decreasing feed conversion ratio), and/or increasing milk fat yield, of such animals.

### BACKGROUND OF THE INVENTION

Antimicrobial agents have been used as antibiotic growth promoters in livestock feed for many decades. However, resistance to such antimicrobial agents is now considered a serious threat to human health. The use of antimicrobial agents as a growth promoter in livestock feed has been fully banned in the European Union since January 2006. At the same time, the demand for protein from animal origin is increasing, fuelled by the human population increase. Additionally, the food production needs to be more sustainable, requiring that more is produced with less. Taken together, food animal production needs to be more and more effective.

These developments have initiated a surge of exploring feed additives that improve performance of farming animals yet do not induce resistance to bacteria. Phytogenic feed additives, also known as botanicals, are substances of plant origin added to animal diets with the aim of improving animal performance. Essential oils, herbs and spices all serve as sources for bioactive ingredients, e.g. phenols, polyphenols, flavonoids, terpenoids and others.

Glycyrrhiza glabra is a Fabaceae plant from the Leguminosae family, native to Eurasia, to central and south-western Asia and the Mediterranean region. Specifically, the roots (with stolon and rhizome) of Glycyrrhiza glabra are denoted as licorice. They have been used by humans for at least 4000 years in various food applications. The roots of Glycyrrhiza glabra are used as a medicinal plant for a variety of effects (El-Saber Batiha et al. 2020. Biomolecules, 10): treatment of digestive system disorders, respiratory tract disorders, epilepsy, fever, sexual debility, paralysis, rheumatism, leucorrhoea, psoriasis, prostate cancer, malaria, hemorrhagic diseases, and jaundice. Within the roots, glycyrrhizic acid (also known as glycyrrhizin, glycyrrhetinic acid glycoside or 18-β glycyrrhetic acid) is the main constituent. Glycyrrhizic acid represents about 10% of the licorice root dry weight (Pastorino et al. 2018. Phytother Res, 32:2323-39).

In the most recent literature reviews considering licorice, authors are solely discussing the roots part of the plant (Bisht et al. 2022. Phytomed Plus, 2:100206; Hejazi et al. 2021. Food Chem Toxicol, 150:112057). For instance, in a review summarizing the anti-inflammatory effect of licorice, especially at the intestinal level, Leite et al. (2022. Int J Mol Sci, 23) focused on glycyrrhizin and glycyrrhetinic acid, which are both compounds only found in the roots of Glycyrrhiza glabra.

Licorice powder and licorice essential oils has been previously used in farming animals. Alagawany et al. (2019. Animals (Basel), 9) summarized the interest of licorice powder to improve performance of poultry, focusing on glycyrrhizic acid, a compound exclusively present in the roots. Thus, in the art, licorice equates with the roots of the Glycyrrhiza glabra plant.

Vlaisavljević et al. (2018. Industrial Crops and Products, 112:217-24) compared 2 samples of roots and 2 samples of leaves of Glycyrrhiza glabra using different chromatography techniques and showed that glycyrrhizic acid was only detected in the root extracts, not in the leaves. They also reported radically different composition between roots and leaves. Siracusa et al. (2011. Fitoterapia, 82:546-56) demonstrated that compounds of the Glycyrrhiza glabra leaves are either present in small traces or not at all in the roots of the same plant. Aerial parts of Glycyrrhiza glabra, such as leaves, are considered an agrochemical waste (Pastorino et al. 2018. Supra. Siracusa et al. 2011. Supra. Gowthaman et al. 2021. Letters in Animal Biology 01(2): 14-20). They do not contain glycyrrhizic acid, which is considered the most important compound present in the roots. Zamiri et al. (January 2015. Trop Anim Health Prod) used Glycyrrhiza glabra leaves as forage as a source of so-called “condensed tannins” for lambs. Ashraf et al. (2017. Pak J Pharm Sci, 30:567-72) injected Glycyrrhiza glabra leaves extract into eggs to study its effect on Newcastle disease virus. The authors suggested that Glycyrrhiza glabra leaves extract could be used to control Newcastle disease virus. Abarghuei and Salem (2021. Environ Sci Pollut Res Int) aimed to evaluate the effect of pulp and leaves of Glycyrrhiza glabra to reduce the ruminal biogas production in sheep.

It is an object of the present invention to provide a feed additive and/or an animal feed to improve performance of animals, preferably farming animals, preferably of many different species of farming animals, e.g., for increasing feed intake, increasing body weight gain, increasing average daily gain, increasing feed efficiency (i.e., decreasing feed conversion ratio), increasing specific growth rate, increasing relative growth rate, increasing milk fat yield, and/or for preventing, reducing the severity of, and/or treating microbial infection, e.g. bacterial infection, viral infection, parasitic infection, fungal infection, and/or yeast infection, in such animals, and/or for preventing, reducing the severity of, and/or treating liver toxicity or liver damage due to mycotoxin exposure.

### SUMMARY OF THE INVENTION

The present disclosure provides an animal feed comprising Glycyrrhiza glabra aerial parts, preferably Glycyrrhiza glabra leaves, or an extract thereof.

In an embodiment, the Glycyrrhiza glabra aerial parts, preferably Glycyrrhiza glabra leaves, are dried, and optionally ground.

In an embodiment, the Glycyrrhiza glabra aerial parts, preferably Glycyrrhiza glabra leaves, are included in an amount of about 1 mg/kg to about 10000 mg per kg of feed, or an equivalent amount of extract.

In an embodiment, the extract is selected from the group consisting of an aqueous extract, ethanolic extract, methanolic extract, isopropanolic extract, ethylacetate extract, acetonic extract, hexane extract, or a supercritical CO2 extract, or a mixture of any of these.

In a further aspect, the present disclosure is concerned with use of Glycyrrhiza glabra aerial parts, preferably Glycyrrhiza glabra leaves, or an extract thereof, as a feed additive. In an embodiment, said feed additive is supplied with written instructions to include it into animal feed in an amount of Glycyrrhiza glabra aerial parts, preferably Glycyrrhiza glabra leaves, of about 1 mg/kg to about 10000 mg per kg of feed, or an equivalent amount of extract.

In an embodiment, the feed additive is included in a premix.

In yet another aspect, the present disclosure provides use of Glycyrrhiza glabra aerial parts, preferably Glycyrrhiza glabra leaves, or an extract thereof, for increasing performance of an animal, e.g., for increasing feed intake, increasing average daily gain, increasing feed efficiency (i.e., decreasing feed conversion ratio), increasing relative growth rate, increasing specific growth rate, and/or increasing milk fat yield.

The present disclosure further pertains to a composition comprising Glycyrrhiza glabra aerial parts, preferably, Glycyrrhiza glabra leaves, or an extract thereof, or an animal feed as taught herein for use in reducing mortality, and/or for use in preventing, alleviating the severity of, and/or reducing microbial infection. In an embodiment, the microbial infection is caused by a Gram-negative bacteria, e.g., of the genus Vibrio, e.g., of the species Vibrio parahaemolyticus.

In an embodiment, the extract is selected from the group consisting of an aqueous extract, ethanolic extract, methanolic extract, isopropanolic extract, ethylacetate extract, acetonic extract, hexane extract, or a supercritical CO2 extract, or a mixture of any of these.

In an embodiment, the feed additive is intended for inclusion in feed for farming animals or companion animals.

In an embodiment, the farming animals are selected from the group consisting of poultry, swine, ruminants, e.g., beef cattle and dairy cattle, fish, e.g. salmon, trout, seabream, sea brass, tilapia, tuna, and the like, and crustaceans, e.g., shrimp.

In another embodiment, the companion animal is selected from the group consisting of ornamental fish, cats, dogs, horses, rabbits, guinea pigs, and hamsters.

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references cited herein are incorporated herein in their entirety by reference.

The term “licorice” (or “liquorice”) is the common name of Glycyrrhiza glabra. In the prior art, the term is used to refer to the roots of the plant.

The term “aerial part” when referring to a plant refers to anything that exists in the air or in the space above a solid surface. The aerial part of plants simply denotes the structures of a plant that are above ground, including the stems, leaves, petioles, flowers, fruits and seeds. The term includes any aerial part alone, e.g., leaves only, or stem only, or the entire aerial part (i.e., all plant parts above the ground). In an embodiment, when the term “aerial parts” is used herein, it can be replaced with the term “leaves”.

The term “leaves” when referring to a plant refers to any usually flattened green outgrowth from the stem of a vascular plant. As the primary sites of photosynthesis, leaves manufacture food for plants, which in turn ultimately nourish and sustain all land animals. Botanically, leaves are an integral part of the stem system. They are attached by a continuous vascular system to the rest of the plant so that free exchange of nutrients, water, and end products of photosynthesis (oxygen and carbohydrates in particular) can be carried to its various parts.

The term ‘farming animal’ refers to animals that are kept or raised for agricultural purposes, like for consumption or to generate income by for example wool, meat, eggs or milk products. Farming animals can be grouped based on their digestive system. Such groups include monogastric, ruminant and pseudo-ruminant animals. Alternatively or additionally, they may be grouped based on their ecosystem. Such groups include aquaculture animals (when referring to animals that are reared in an aqueous ecosystem such as fish and shrimp) and livestock animals (when referring to animals that are reared on land such as poultry, swine, and bovines).

The term “companion animal” refers to domesticated or domestic-bred animals whose physical, emotional, behavioral and social needs can be readily met as companions in the home, or in close daily relationship with humans. Species suitable to be companion animals include dogs, cats, horses, rabbits, ferrets, birds, guinea pigs and select other small mammals, small reptiles and ornamental fish.

The term “pellets” or “feed pellets” as used herein refers to small particles or a body typically created by compressing an original material, for instance a mixture of raw feed material, typically fermentable feed ingredients such as grains, cereals, legumes, roughage, and the likes. Feed pellets may also comprise other feed ingredients such as meat meal, fish meal, bone meal, by-process products, oil, fat, fillers or any mixture thereof, etc., as well as minerals, vitamins and trace elements and others. Animal feed pellets vary in their composition as well as structural properties (e.g. hardness, density, durability, shape, size, etc.) depending on the nutritional needs, eating habits, digestive system (monogastric system, ruminant digestive system, etc.) and habitat (e.g., aquatic, terrestrial, domestic, etc.) of the animal for which the feed pellet is intended. Animal feed pellet may have any size, shape (e.g. round, rectangular, cylindrical, etc), weight and/or length. It is understood that the weight of the feed pellet will depend on the feed pellet composition per se (e.g. some ingredients have a greater weight or density than others) as well as the shape, size and length of the finished feed pellet product. It is known that the size, shape, weight and/or length of the feed pellet will influence pellet durability. This is true for any method for making feed pellets, including method as taught herein. It is further commonly agreed in the field of agriculture and animal nutrition that animals (e.g. young and adult livestock animals like beef (e.g. beef calf), dairy cows (e.g. dairy calf), and pigs, etc.) benefit more or make better gains (e.g. weight gain, increased height, enhanced growth curve) on pelleted feed than a meal ration because pelleted feed is in a more concentrated, readily edible and palatable form than meal or mash ration. Pelleted feed has been shown to ease food intake and minimize feed waste during the eating process. It was shown that most animals, if given the choice between the same feed in a pellet or a mash form, will prefer the pellet form. Animal feed pellets are typically produced on an industrial scale using for example a pelleting process. The skilled person is well-acquainted with processes for producing animal feed pellets.

The term “premix” as used herein refers to a mixture of ingredients designed to be mixed with other ingredients, usually raw materials to be used for feed, before use. The ingredients within the mixture of ingredients within the premix are usually ingredients which are to be added to animal feed in small quantities, such as vitamins and minerals, and optionally also antioxidants, pigments, and/or organic acids. For example, a premix for fish feed may comprise vitamins, minerals, antioxidants and/or pigments, but does not usually comprise organic acids. In contrast, a premix for poultry or swine feed may comprise vitamins, minerals, and organic acids, and antioxidants, but not pigments.

The term “feed conversion ratio” or “FCR” as used herein is a ratio or rate measuring of the efficiency with which the bodies of farming animals convert animal feed into the desired output, e.g., weight. The term “feed efficiency” as used herein is the inverse of the FCR.

The term “relative growth rate” or “RGR” as used herein is expressed in percentage and is calculated as follows: (Final weight-initial weight)/initial weight.

The term “specific growth rate” or “SGR” is a coefficient that measures the percentage increase in fish weight per day: SGR=(Ln(Wt)−Ln(W0))*100/t (d), wherein W0[g]=the weight in grams at the beginning of the period, Wt [g]=the weight in grams at the end of the period, t[d]=period, expressed in number of days; and Ln=natural logarithm.

The term “secondary plant constituents” as used herein refers to specialized compounds present in plants that do not aid in the growth and development of plants but are required for the plant to survive in its environment. Such secondary plant constituents may be essential for communicating with other organisms in mutualistic (e.g. attraction of beneficial organisms such as pollinators) or antagonistic interactions (e.g. deterrent against herbivores and pathogens). They may further assist in coping with abiotic stress such as increased UV-radiation. The broad functional spectrum of specialized metabolism is still not fully understood.

The term ‘about’, as used herein indicates a range of normal tolerance in the art, for example within 2 standard deviations of the mean. The term “about” can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”.

Reference to an element by the indefinite article “a” or “an” does not exclude the possibility that more than one of the elements is present, unless the context clearly requires that there be one and only one of the elements. The indefinite article “a” or “an” thus usually means “at least one”.

### BRIEF DESCRIPTION OF THE FIGURE

FIG. 1 compares the chromatograms of Glycyrrhiza glabra root sample (clearly showing the Glycyrrhizic acid peaks) with Glycyrrhiza glabra leaf sample where the glycyrrhizic acid peaks are absent.

FIG. 2 shows fecal oocyst shedding of chickens without (NC) or with (PC, MO, GG) E. maxima infection in the absence of further treatment (PC) or with treatment using monensin (MO), 55 ppm Glycyrrhiza glabra leaves (55 ppm GG) or 160 ppm Glycyrrhiza glabra leaves (160 ppm GG).

### DETAILED DESCRIPTION OF THE INVENTION

This patent application has been drafted into sections. However, these sections should not be read in isolation. Unless otherwise specified, each section is to be read in combination with the other sections. This means, for example, that all of the animal feeds and/or feed additive compositions described in the “Animal feed and/or feed additive composition” section are intended to be read with the “Methods of using the animal feed and/or feed additive” section (i.e., all animal feeds recited in the first section are suitable for the methods described in the latter section). The various optional and preferred features can also be combined, even when taken from different parts of the specification. Likewise, all “aspects” and “embodiments” can be combined. No separation of embodiments is intended, unless explicitly stated.

The present inventors have surprisingly found that inclusion in a relatively small amount of Glycyrrhiza glabra aerial parts, preferably leaves, in broiler feed, shrimp feed, or salmon feed, increases their performance, e.g., in terms of body weight gain or average daily gain and feed efficiency.

Animal Feed and/or Feed Additive Composition

Thus, the present disclosure provides an animal feed comprising Glycyrrhiza glabra aerial parts, or an extract thereof, preferably Glycyrrhiza glabra leaves, or an extract thereof.

The animal feed may be in any form suitable for ingestion by the relevant animal. For example, poultry feed may be in the form of crumble, mash, or pellets. Swine feed may be in the form of meal or pellets. Feed for fish and crustaceans is usually in the form of extruded, e.g., cooking extruded, or pressed pellets but may also be in another form, e.g., in the form of flakes. In an embodiment, the animal feed may be in the form of a crumble, mash, meal, flakes, or pellets, which pellets may be extruded, e.g., cooking extruded, or may be pressed. In a suitable embodiment, the animal feed is in the form of a pellet.

The animal feed generally comprises protein, fat, carbohydrates, minerals and vitamins. It may further comprise antioxidants, pigments, organic acids, enzymes, phytogenic compounds, and/or other feed additives. The skilled person knows how to formulate a suitable animal feed depending on the target animal, life stage of the target animal, and/or physical condition of the target animal.

The Glycyrrhiza glabra aerial parts, preferably leaves, may be from any Glycyrrhiza glabra variety or subspecies. Optionally, the aerial parts or leaves may have been processed, e.g., fermented, prior to their use in animal feed or a feed additive as taught herein.

The Glycyrrhiza glabra aerial parts, preferably Glycyrrhiza glabra leaves, may be used fresh or may be dried by any means known in the art. The skilled person knows suitable drying technologies. For example, drying may take place using a drum drier, belt dryer, dehydrator drying, vacuum drying, microwave drying, or infrared-drying, or natural drying (sun drying). In a preferred embodiment, the Glycyrrhiza glabra aerial parts or Glycyrrhiza glabra leaves are dried.

Prior to or after drying, preferably after drying, the Glycyrrhiza glabra aerial parts, preferably Glycyrrhiza glabra leaves, may be ground to any suitable size. For example, the fresh or dried leaves may be ground to an average particle size of smaller than 2 mm, preferably smaller than 1.5 mm, even more preferably smaller than 1 mm, yet more preferably smaller than 0.8 mm, even more preferably smaller 0.6 mm, most preferably smaller than 0.5 mm, smaller than 0.4 mm or smaller than 0.3 mm, or even smaller than 0.2 mm or 0.150 mm.

The Glycyrrhiza glabra aerial parts, preferably Glycyrrhiza glabra leaves, may be included in an amount of about 1 mg/kg to about 10000 mg/kg of feed, preferably about 5 mg/kg to about 8000 mg/kg of feed, more preferably about 10 mg/kg to about 6000 mg/kg of feed, even more preferably about 15 mg/kg to about 4000 mg/kg of feed, yet more preferably about 20 mg/kg to about 3000 mg/kg of feed, again more preferably about 25 mg/kg to about 2000 mg/kg of feed, such as about 30 mg/kg to about 1500 mg/kg of feed, about 40 mg/kg to about 1200 mg/kg of feed, about 50 mg/kg to about 1000 mg/kg of feed, or about 60 mg/kg to about 800 mg/kg of feed.

In an embodiment, the animal feed taught herein may be a feed suitable for ingestion by livestock animals, e.g., poultry, swine, or ruminants such as dairy cows, beef cattle, sheep, goats, and the like. The skilled person knows how to formulate a feed suitable for the particular species of livestock as well as the life stage of the particular species of livestock.

In an embodiment, the animal feed taught herein may be an aquaculture feed, i.e., a feed suitable for ingestion by fish or crustaceans. Said feed may preferably be in the form of a pellet, more preferably an extruded, e.g., cooking extruded, or a pressed pellet. Said feed may comprise protein, fat, carbohydrates, vitamins, minerals, and water, and may optionally further comprise pigments, e.g., astaxanthin, and/or antioxidants. The exact amounts of protein, fat, carbohydrates, vitamins, minerals, and water may depend on the species of fish or crustacean as well as the life stage of the fish or crustacean that the feed is to be fed to. The skilled person knows how to formulate a feed for fish or crustaceans of a suitable composition.

The feed may, when it is produced to cover the nutritional needs for marine fish such as cod, pollock, sea bass and sea bream, contain at least 10%, at least 12.5%, or at least 15% fat by weight. Alternatively, the feed may contain at least 18%, at least 20%, at least 23%, at least 25%, at least 26%, or at least 28% by weight fat when it is intended to be fed to salmonids. Preferably, the fat used in feed suitable for fish or crustaceans may comprise w3-fatty acids such DHA and/or EPA.

In an embodiment, the animal feed taught herein may be a feed suitable for a companion animal, e.g., dog, cat, horse, etcetera. The skilled person knows how to formulate a feed for companion animals depending on the type of companion animal, as well as its life stage.

Alternatively to aerial parts or leaves, or additionally, an equivalent amount of extract of Glycyrrhiza glabra aerial parts or Glycyrrhiza glabra leaves may be used.

Plant extraction is a process that aims to extract certain components (so-called secondary plant constituents, including, without limitation, alkaloids, terpenoids, saponins, phenolic compounds, flavonoids, and/or tannins) present in plants. It is a solid/liquid separation operation: a solid object (in the present case Glycyrrhiza glabra aerial parts, preferably Glycyrrhiza glabra leaves, optionally dried and/or ground) is placed in contact with a fluid (the solvent). The plant components of interest are then solubilised and contained within the solvent. The solution thus obtained is the desired extract. The solvent may eventually be eliminated, although this is not required. The solvent may be any solvent suitable for preparing plant extracts for use in animal feed. Suitable solvents include, without limitation, polar solvents (e.g., water, alcohols such as ethanol, methanol and isopropanol), intermediate polar solvent (e.g., acetone, dichloromethane), and nonpolar solvents (e.g., ethylacetate, hexane, ether, chloroform). The skilled person may select a single solvent or two or more solvents to prepare an extract in accordance with the present disclosure. In general, extraction procedures include maceration, digestion, decoction, infusion, percolation, Soxhlet extraction, supercritical extraction, ultrasound-assisted, and microwave-assisted extractions, supercritical fluid extraction (e.g. with supercritical carbon dioxide), accelerated solvent extraction, and solid-phase extraction. Fractionation and purification of phytochemical substances may be achieved through application of various techniques such as, without limitation, liquid-liquid partitioning, cross-flow filtration techniques and chromatographic methods, such as paper chromatography, thin-layer chromatography, gas chromatography, and high-performance liquid chromatography, and centrifugal partitioning chromatography. Finally, compounds obtained are characterized using diverse identification techniques such as mass spectroscopy, infrared spectroscopy, ultraviolet spectroscopy, and nuclear magnetic resonance spectroscopy, or any combination thereof. The skilled person is capable of selecting a suitable extraction method.

The amount of extract of Glycyrrhiza glabra aerial parts or Glycyrrhiza glabra leaves to be incorporated into the animal feed and/or feed additive taught herein may be determined by equating the content of one or more secondary plant constituents in the extract to the content of said one or more secondary plant constituents of the indicated amounts of Glycyrrhiza glabra aerial parts or Glycyrrhiza glabra leaves. An amount of extract that provides a content of one or more secondary plant constituents that is equivalent to the content of said one or more secondary plant constituents in the indicated amount of Glycyrrhiza glabra aerial parts or leaves may thus be incorporated into said animal feed. For example, secondary plant constituents present in Glycyrrhiza glabra leaves include, without limitation, phenolic compounds, e.g., flavanones, prenylated dyhydrostilbenes, prenylisoflavones, prenylated flavanon, dihydrochalchones, chalcones, flavone-C-glycosides, flavonol-O-glycosides, hydroxy-cinnamic acids, and coumarins in their glycosidic form or as aglycon.

In an embodiment, the extract is selected from the group consisting of an aqueous extract, ethanolic extract, methanolic extract, isopropanolic extract, ethylacetate extract, acetonic extract, hexane extract, or a supercritical CO2 extract, or a mixture of any of these. Alternatively, a mixture of solvents may be used for extraction, as set forth above.

The present disclosure further provides a feed additive comprising Glycyrrhiza glabra aerial parts, preferably Glycyrrhiza glabra leaves, or an extract thereof, as taught herein. Moreover, the present disclosure provides use of Glycyrrhiza glabra aerial parts, preferably Glycyrrhiza glabra leaves, or an extract thereof, as a feed additive.

Said feed additive may further comprise vitamins, minerals, organic acids, antioxidants, and/or pigments. Said feed additive may be provided in the form a premix. The feed additive is preferably intended for inclusion in feed for farming animals or companion animals.

Said feed additive may be supplied with written instructions to include it into animal feed in an amount of Glycyrrhiza glabra aerial parts, preferably Glycyrrhiza glabra leaves, of about 1 mg/kg to about 10000 mg/kg of feed, preferably about 5 mg/kg to about 8000 mg/kg of feed, more preferably about 10 mg/kg to about 6000 mg/kg of feed, even more preferably about 15 mg/kg to about 4000 mg/kg of feed, yet more preferably about 20 mg/kg to about 3000 mg/kg of feed, again more preferably about 25 mg/kg to about 2000 mg/kg of feed, such as about 30 mg/kg to about 1500 mg/kg of feed, about 40 mg/kg to about 1200 mg/kg of feed, about 50 mg/kg to about 1000 mg/kg of feed, or about 60 mg/kg to about 800 mg/kg of feed, or, in case an extract of Glycyrrhiza glabra aerial parts or Glycyrrhiza glabra leaves is used, an equivalent amount of extract as taught above.

Methods of Using the Animal Feed and/or Feed Additive

In an aspect, the present disclosure provides a method of feeding an animal, e.g., a farming animal, such as a chicken, swine, cow, fish, shrimp, or the like, or a companion animal, such as a dog or a cat, with an animal feed as taught herein.

In a further aspect, the present disclosure relates to use of Glycyrrhiza glabra aerial parts, preferably Glycyrrhiza glabra leaves, or an extract thereof, or animal feed or feed additive as taught herein, for increasing performance of an animal.

In another aspect, the present disclosure is concerned with use of Glycyrrhiza glabra aerial parts, preferably Glycyrrhiza glabra leaves, or an extract thereof, or an animal feed or feed additive as taught herein, for increasing feed intake, increasing body weight gain, increasing average daily gain, increasing feed efficiency (i.e., decreasing feed conversion ratio), increasing relative growth rate, increasing specific growth rate, and/or increasing milk fat yield.

In an embodiment, the Glycyrrhiza glabra aerial parts, preferably Glycyrrhiza glabra leaves, or an extract thereof, or a feed additive or animal feed as taught herein, may be used as an antibacterial agent, as an antiviral agent, as an antifungal agent, or as an antiparasitic agent.

In another embodiment, the Glycyrrhiza glabra aerial parts, preferably Glycyrrhiza glabra leaves, or an extract thereof, or a feed additive as taught herein may be used as a preservative agent or as a cytoprotective agent.

Further, the present disclosure provides use of a composition comprising Glycyrrhiza glabra aerial parts, preferably, Glycyrrhiza glabra leaves, or an extract thereof, as taught herein, or an animal feed or feed additive as taught herein in reducing mortality, e.g., in disease challenged animals, e.g. in shrimp challenged with a disease, e.g., challenged with acute hepatopancreatic necrosis disease (AHPND), e.g., by bacteria of the Vibrio genus, e.g. of the species Vibrio parahaemolyticus.

The present disclosure further provides use of a composition comprising Glycyrrhiza glabra aerial parts, preferably, Glycyrrhiza glabra leaves, or an extract thereof, as taught herein, or an animal feed or feed additive as taught herein in preventing, alleviating the severity of, and/or reducing microbial infection and/or diseases caused by such microbial infection.

The present disclosure also provides a method of preventing, alleviating, and/or reducing microbial infection in an animal, said method comprising the step of administering to said animal a composition comprising Glycyrrhiza glabra aerial parts, preferably, Glycyrrhiza glabra leaves, or an extract thereof, or an animal feed or feed additive as taught herein. The microbial infection may be caused by any microbe, such as a bacterium, virus, parasite, yeast or fungus.

The microbial infection may, e.g., be caused by Gram positive or Gram negative bacteria.

Non-limiting examples of bacteria that may be causing the microbial infection include Escherichia species, Salmonella species, Vibrio species, Flavobacterium species, Campylobacter species, Clostridium species, Streptococcus species, Piscirickettsia species such as Piscirickettsia salmonis causing Salmon Rickettsial Syndrome (SRS), and others, such as Escherichia coli, Salmonella typhimurium, Salmonella dublin, Salmonella enterica, Salmonella enteritidis, Clostridium perfringens, Pasteurella multocida, Listeria monocytogenes, Mycoplasma bovis, Haemophilus somnus, Campylobacter jejunum, Campylobacter hepaticus, Campylobacter bilis, and the like.

In an embodiment, the microbial infection is caused by a Gram-negative bacteria, e.g., of the genus Vibrio.

In aquaculture, several Vibrio spp. are currently considered pathogens or opportunistic pathogens of reared finfish, shellfish, and shrimp, among which V. anguillarum, V. ordalii, V. vulnificus, V. alginolyticus, V. parahaemolyticus, Aliivibrio salmonicida, V. harveyi, V. tubiashii, and V. cholerae. Vibriosis caused by the abovementioned species is a common and devastating bacterial disease in fish larviculture and aquaculture.

In an embodiment, the microbial infection is caused by a microbe of the species V. anguillarum, V. ordalii, V. vulnificus, V. alginolyticus, V. parahaemolyticus, Aliivibrio salmonicida, V. harveyi, V. tubiashii, and/or V. cholerae. In such case, the animal infected may be a fish or crustacean, e.g., a shrimp. In an embodiment, the microbial infection is caused by V parahaemolyticus. The infected animal may be a crustacean, e.g., a shrimp. The diseases caused by infection with V. parahaemolyticus may be Vibriosis.

Other Gram-negative bacteria that may be causing the microbial infection may be members of the Flavobacteriaceae, e.g., of the genus Flavobacterium, for example those selected from the group consisting of F. columnaris, F. johnsoniae, Tenacibaculum maritimum (formerly known as Flexibacter maritimus), F. psychrophilum, F. branchiophilum, Tenacibaculum ovolyticum (formerly Flexibacter ovolyticus), and Chryseobacterium scopthalmum (formerly F. scopthalmum). The diseases caused by infection with such bacteria may be Flavobacteriosis.

In another embodiment, the microbial infection is caused by a Gram-positive bacterium, e.g. of the genus Streptococcus, e.g., Streptococcus agalactiae.

Members of the genus Streptococcus cause mild to severe bacterial illnesses in animals. These organisms typically colonize one or more species as commensals and can cause opportunistic infections in those hosts. S. agalactiae is a common cause of subclinical mastitis in cattle. In some regions, it also seems to be fairly common in clinical or subclinical mastitis in sheep and goats. S. canis is an opportunistic pathogen that mainly affects dogs and cats. It can cause a variety of diseases including skin and soft tissue infections, arthritis, reproductive disease, mastitis, pneumonia, septicemia and streptococcal toxic shock-like syndrome, as well as cervical lymphadenitis in 3-6 month old kittens and otitis externa in dogs. In cats, this organism sometimes causes neonatal septicemia. S. dysgalactiae subsp. Dysgalactiae is usually associated with clinical or subclinical mastitis in cattle, but it has been detected in other conditions in this species, including severe cellulitis and toxic shock-like syndrome. Some reports of S. dysgalactiae subsp. Dysgalactiae in other hosts described mastitis in sheep and goats, suppurative polyarthritis in lambs, septicemia in fish and dogs, and septicemia and encephalitis in vampire bats.

In an embodiment, the bacterium of the genus Streptococcus is selected from the group consisting of Streptococcus agalactiae, Streptococcus canis, Streptococcus dysgalactiae subsp. Dysgalactiae, Streptococcus equi subsp. Zooepidemicus, Streptococcus halichoeri, Streptococcus iniae, and Streptococcus suis. The diseases caused by infection with such bacteria may be skin and soft tissue infections, arthritis, reproductive disease, mastitis, pneumonia, septicemia and streptococcal toxic shock-like syndrome.

Non-limiting examples of viruses that may cause the microbial infection include, depending on the type of farming animal or companion animal, white spot syndrome virus (WSSV) causing white spot syndrome or white spot disease, Infectious Hypodermal and Hematopoietic Necrosis Virus (IHHNV) causing Infectious Hypodermal and Hematopoietic Necrosis, Taura Syndrome Virus (TSV) causing Taura Syndrome, Infectious Myonecrosis Virus (Myo/IMNV) causing Infectious Myonecrosis, Piscine myocarditis virus (PMCV) causing Piscine myocarditis, viral haemorrhagic septicaemia virus causing viral haemorrhagic septicaemia, infectious haematopoietic necrosis virus causing infectious haematopoietic necrosis, infectious salmon anaemia virus (ISAV) causing infectious salmon anaemia, piscine orthoreovirus (PRV) causing Heart and Skeletal Muscle Inflammation (HSMI), jaundice syndrome, proliferative darkening syndrome and erythrocytic body inclusion syndrome in fish, Tilapia lake virus, Covert mortality nodavirus, Shrimp hemocyte iridescent virus, and Abalone herpesvirus, Porcine reproductive and respiratory syndrome virus (PRRSV) causing Porcine reproductive and respiratory syndrome, porcine epidemic diarrhea virus (PEDV) causing porcine epidemic diarrhea, African swine fever virus causing, African swine fever, Classical swine fever virus causing Classical swine fever, Nipah virus, Swine vesicular disease virus causing swine vesicular disease, Transmissible gastroenteritis virus of swine causing transmissible gastroenteritis, avian influenza viruses causing avian influenza, Infectious bursal disease virus causing infectious bursal disease, Marek's disease virus causing Marek's disease, Avian metapneumovirus, Avian infectious bronchitis virus causing avian infectious bronchitis, Infectious laryngotracheitis virus causing infectious laryngotracheitis, Duck hepatitis virus causing duck hepatitis, Pseudorabies virus causing pseudorabies, Bluetongue virus causing bluetongue virus, Foot-and-mouth disease virus (serotypes A, O, C, SAT1, SAT2, SAT3, Asia1) causing Foot-and-mouth disease, Japanese encephalitis virus causing Japanese encephalitis, Rabies virus causing rabies, Rift Valley fever virus causing Rift Valley fever, Rinderpest virus causing Rinderpest, Vesicular stomatitis virus causing Vesicular stomatitis, West Nile fever virus causing West Nile fever, and others.

In an embodiment, the microbial infection is caused by a parasite, e.g., an endoparasite or an ectoparasite. The parasite may be a member of the phylum Apicomplexa that includes Cryptosporidium spp., Plasmodium spp., Eimeria spp., Neospora, Babesia, and Theileria.

Non-limiting examples of parasites taught herein include those of the genus Eimeria causing coccidiosis in a variety of animal species, those of the genus Cryptosporidium causing cryptosporidiosis, and those of the genus Enterocytozoon causing, a.o., early mortality syndrome (EMS) or acute hepatopancreatic necrosis disease (AHPND) in shrimp (Enterocytozoon hepatopenaei).

Parasites of the genus Eimeria include, without limitation, Eimeria tenella, Eimeria acervulina, Eimeria praecox, Eimeria mitis, Eimeria necatrix and Eimeria maxima causing coccidiosis in poultry, Eimeria zuernii, Eimeria alabamensis and Eimeria bovis causing coccidiosis in ruminants such as cattle, as well as any other Eimeria species that cause coccidiosis in various animal species.

Parasites of the genus Cryptosporidium include, without limitation, Cryptosporidium parvum, Cryptosporidium meleagridis, Cryptosporidium felis, Cryptosporidium canis and Cryptosporidium hominis all causing cryptosporidiosis.

The present disclosure further provides a method of improving performance of animals, for example farming animals, e.g., increasing feed intake, increasing average daily gain, increasing feed efficiency (i.e., decreasing feed conversion ratio), and/or increasing milk yield, said method comprising the step of administering Glycyrrhiza glabra aerial parts, preferably Glycyrrhiza glabra leaves, or an extract thereof, or an animal feed or feed additive as taught herein to said animals.

Further, the present disclosure provides a method of reducing mortality in an animal, said method comprising the step of administering to said animal a composition comprising Glycyrrhiza glabra aerial parts, preferably, Glycyrrhiza glabra leaves, or an extract thereof, or an animal feed or feed additive as taught herein.

In addition, it has been found that the composition taught herein provides cells, in particular liver cell, protection against mycotoxins such as Aflatoxin B1. It is known that absorbed mycotoxins, like all toxins, are detoxified in the liver. Even short-term exposure to unchecked mycotoxins suffices to cause significant liver damage and loss of performance of the liver. Hence, the composition taught herein may also be used as a preservative agent or as a cytoprotective agent. The animal feed or composition taught herein may be used to prevent, reduce the severity of, and/or treat liver toxicity or liver damage due to mycotoxin exposure.

The animal feed and/or feed additive for use in the above methods may be as described hereinabove.

The extract may be as described hereinabove.

The animals may be selected from farming animals and companion animals. The farming animals may be selected from the group consisting of poultry, swine, ruminants, e.g., beef cattle and dairy cattle, fish, e.g. salmon, trout, seabream, sea brass, tilapia, tuna, and the like, and crustaceans, e.g., shrimp. The companion animals may be selected from the group consisting of ornamental fish, cats, dogs, horses, rabbits, guinea pigs, and hamsters.

The animals may be animals that are affected by a microbial infection (also referred to as “infected animal”) or that are at risk of being infected by a microbe (also referred to as “the animal at risk of infection”). The infected animal, or the animal at risk of infection may be an aquatic species, e.g., a fish or a shrimp, or may be a livestock species, e.g., chicken, swine, or cow, or any other animal species referred to herein.

In an embodiment, the benefits in performance obtained in animals fed the feed additive or animal feed taught herein are relative to results obtained with animals (of the same species) fed an identical animal feed yet without such feed additive.

The present invention is further illustrated, but not limited, by the following examples. From the above discussion and the examples, one skilled in the art can ascertain the essential characteristics of the present invention, and without departing from the teaching and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Suitable Embodiments

1. Animal feed comprising Glycyrrhiza glabra aerial parts, preferably Glycyrrhiza glabra leaves, or an extract thereof, wherein the Glycyrrhiza glabra aerial parts, preferably Glycyrrhiza glabra leaves, are included in an amount of about 1 mg/kg to about 10 g per kg of feed, or an equivalent amount of extract.
2. Animal feed according to embodiment 1, wherein the Glycyrrhiza glabra aerial parts, preferably Glycyrrhiza glabra leaves, are dried, and optionally ground.
3. Animal feed according to any one of embodiments 1 or 2, wherein the extract is selected from the group consisting of an aqueous extract, ethanolic extract, methanolic extract, isopropanolic extract, ethylacetate extract, acetonic extract, hexane extract, or a supercritical CO2 extract, or a mixture of any of these.
4. Use of Glycyrrhiza glabra aerial parts, preferably Glycyrrhiza glabra leaves, or an extract thereof, as a feed additive, wherein said feed additive is supplied with written instructions to include it into animal feed in an amount of Glycyrrhiza glabra aerial parts, preferably Glycyrrhiza glabra leaves, of about 1 mg/kg to about 10000 mg per kg of feed, or an equivalent amount of extract.
5. Use of Glycyrrhiza glabra aerial parts, preferably Glycyrrhiza glabra leaves, or an extract thereof, as an antibacterial, antiviral, antifungal, anti-yeast agent, antiparasitic or feed preservative agent.
6. Use according to embodiment 4 or 5, wherein the feed additive, antibacterial, antiviral, antifungal, anti-yeast agent, antiparasitic or feed preservative agent is included in a premix.
7. Method of feeding an animal, said method comprising the step of administering to said animal an animal feed according to any of embodiments 1-3.
8. Use of Glycyrrhiza glabra aerial parts, preferably Glycyrrhiza glabra leaves, or an extract thereof, or an animal feed according to any one of embodiments 1-3 for increasing performance of an animal.
9. Use of Glycyrrhiza glabra aerial parts, preferably Glycyrrhiza glabra leaves, or an extract thereof, or an animal feed according to any one of embodiments 1-3 for increasing feed intake, increasing average daily gain, increasing feed efficiency (i.e., decreasing feed conversion ratio), increasing relative growth rate, increasing specific growth rate, and/or increasing milk fat yield.
10. A composition comprising Glycyrrhiza glabra aerial parts, preferably, Glycyrrhiza glabra leaves, or an extract thereof, or an animal feed according to any of embodiments 1-3 for use in reducing mortality.
11. A composition comprising Glycyrrhiza glabra aerial parts, preferably, Glycyrrhiza glabra leaves, or an extract thereof, or an animal feed according to any of embodiments 1-3 for use in preventing, alleviating the severity of, and/or reducing microbial infection.
12. A composition for use according to embodiment 11, wherein said microbial infection is caused by a microbe selected from the group consisting of a bacterium, a virus, a fungus, a yeast, or a parasite.
13. A composition for use according to any one of embodiments 11 or 12, wherein the microbial infection is caused by a Gram-negative bacteria, e.g., of the genus Vibrio, e.g., of the species Vibrio parahaemolyticus.
14. A composition for use according to any one of embodiments 11 or 12, wherein the microbial infection is caused by a virus, e.g., white spot syndrome virus.
15. A composition for use according to any one of embodiments 11 or 12, wherein the microbial infection is caused by a parasite, e.g., of the genus Eimeria, of the genus Cryptosporidium or of the genus Enterocytozoon.
16. A composition comprising Glycyrrhiza glabra aerial parts, preferably, Glycyrrhiza glabra leaves, or an extract thereof, or an animal feed according to any of embodiments 1-3 for use in preventing, reducing the severity of, and/or treating liver toxicity or liver damage due to mycotoxin exposure, e.g., exposure to Aflatoxin B1.
17. Use according to any of embodiments 4-6, or composition for use according to any of embodiments 10-16, wherein the extract is selected from the group consisting of an aqueous extract, ethanolic extract, methanolic extract, isopropanolic extract, ethylacetate extract, acetonic extract, hexane extract or mixtures of these solvents or a supercritical CO2 extract, or a mixture of any of these.
18. Use according to any one of embodiments 4-6 or composition for use according to any one of embodiments 10-17, wherein the feed additive is intended for inclusion in feed for farming animals or companion animals.
19. Use according to embodiment 18, wherein the farming animals are selected from the group consisting of poultry, swine, ruminants, e.g., beef cattle and dairy cattle, fish, e.g. salmon, trout, seabream, sea brass, tilapia, tuna, and the like, and crustaceans, e.g., shrimp.
20. Use according to embodiment 18, wherein the companion animal is selected from the group consisting of ornamental fish, cats, dogs, horses, rabbits, guinea pigs, and hamsters.

### EXAMPLES

### Example 1

In this example, a sample of roots of Glycyrrhiza glabra and a sample of leaves of Glycyrrhiza glabra were analyzed for the presence of 18-beta Glycyrrhizic acid, which is typical of the roots of Glycyrrhiza glabra and the main reason for the use of licorice in food and feed applications.

The material used for the analysis was a column Atlantis T3 3.5 μm 4.6×150 mm (No. 1.7) with Acetonitrile and 3% acetic acid in water as solvent.

A reference solution was made by dissolving 10.5 mg monoammonium glycyrrhizate in 0.8% ammonium hydroxide in water and then diluting to 50.0 ml. 25 ml of this stock solution was diluted to 50 ml with 0.8 ammonium hydroxide solution. The stock solution was prepared as follows. Weighting 0.4 g powdered roots and leaves (laboratory mill, sieve 2.0 mm) and extracting them with 50.0 ml 0.8% ammonium hydroxide in water, then for 30 min ultrasonicating and centrifuging for 5 min at 4400 rpm. The supernatant creates the stock solution. The diluted sample solution was prepared as follows. 2 ml of this stock solution was diluted with 0.8% ammonium hydroxide in water to 10 ml and created the diluted sample solution.

The results of the analysis are summarized in Table 1 below.

TABLE 1
18-beta-glycyrrhizic acid content of roots and leaves.

| | 18-beta Glycyrrhizic acid | | |
|---|---|---|---|
| | | | Content related to |
| Sample Name | LoD [%] | Peak area | dry matter [%] |
| Glycyrrhiza glabra | 8.2 | 1046 | 4.13 |
| roots | | | |
| Glycyrrhiza glabra | 9.1 | Not detected | Not quantifiable |
| leaves; diluted | | | |
| Glycyrrhiza glabra | 9.1 | 11 | 0.009 |
| leaves; stock solution | | | |

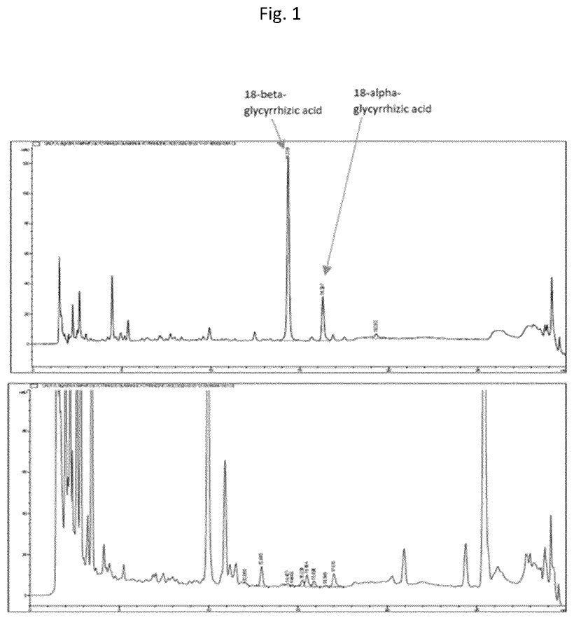

FIG. 1 compares the chromatograms of Glycyrrhiza glabra root sample (top graph, clearly showing the Glycyrrhizic acid peaks) with Glycyrrhiza glabra leaf sample (bottom graph), where the glycyrrhizic acid peaks are absent. Table 1 summarizes the results of FIG. 1 and shows that either no glycyrrhizic acid or trace level of glycyrrhizic acid were detected in the leaves of Glycyrrhiza glabra. Conversely, 4.13% of glycyrrhizic acid was found in the roots sample of the same plant.

This experiment was repeated thrice with leaves of different Glycyrrhiza glabra plants, and the same results were obtained. The typical peaks for glycyrrhizic acid were absent.

### Example 2

The effect of the dietary inclusion doses of Glycyrrhiza glabra leaves on the productive performance of shrimps was investigated.

Glycyrrhiza glabra leaves came from cultivation. Cultivation and drying followed Good Agricultural and Collecting practices. The Glycyrrhiza glabra leaves were grinded to a particle size of <0.3 mm. The basal diet composition was as set forth in Table 2.

TABLE 2
Basal diet composition of extruded shrimp feed.

| | Inclusion levels |
|---|---|
| Ingredients | (in kg/100 kg) |
| Soya products/by-product | 35-40 |
| Wheat products/by-product | 35-40 |
| Fish meal/by-product | 8-15 |
| Macro mineral | 4-6 |
| Soy Lecithin/oil | 1.5-2.5 |
| Fish oil NA | 1.5-2 |
| Crustacean-based attractant | 1.5-3 |
| Hydrolysates | 1.5-3 |
| Amino acids mix | 0.5-1.2 |
| Vitamin-mineral premix | 0.3-0.5 |

The diets were produced as 1.4±0.1 mm extruded pellets.

The feed production procedure followed the normal guidelines for shrimp feed extrusion. First, all dried raw materials (except micro-ingredients) were ground through a 1 mm (50 Hz) grinder screen. Next, ingredients were weighed to a 0.01-g precision on an electronic scale following the formula specifications. After the extrusion, the pellets were dried until the moisture content was below 10%. Final feeds were bagged and stored at 4° C.

The experimental treatments were made as follow. The ‘Control’ shrimps received basal shrimp feed. ‘Treatment’ shrimps received the basal diet supplemented with 520 g per metric ton of feed (520 ppm) of dried ground Glycyrrhiza glabra leaves.

The Control and the Treatment were each assigned to 1 plastic tank. Each tank of a capacity of 290 L was equipped with an individual filtration system and filled with artificial seawater at a salinity of 20 g/L. 75 shrimps were introduced in each tank. Shrimps were Penaeus vannamei postlarvae.

The total weight of the groups was taken at the beginning (day 0) and at the end of the trial (day 18). The mean body weight of the shrimps was calculated at the beginning and at the end for each group. The daily feeding rate for each group was calculated based on shrimp mean body weight and adjusted daily according to the expected shrimp growth and mortality. Feed was distributed automatically 6 times a day during a period of 18 days. Water quality was maintained by the filtration system and regular water changes. Water temperature was kept at 24° C. The room in which the tanks were located was illuminated 12 h per day.

Growth performance was assessed by the following parameters: weight gain (WG) and feed conversion ratio (FCR).

TABLE 3
Performance of shrimps

| Productive response | | | Effect of |
|---|---|---|---|
| (unit) | Control | Treatment | treatment (%) |
| Weight gain (g) | 2.12 | 2.30 | +8.3% |
| FCR | 0.95 | 0.88 | −7.4% |

The results presented in Table 3 show that the addition of Glycyrrhiza glabra leaves in the diet of shrimp improved productive performance. The addition of Glycyrrhiza glabra leaves increased weight gain by 8.3%. Feed conversion ratio was decreased (which means feed efficiency was improved) by the addition of Glycyrrhiza glabra leaves by 7.4%.

### Example 3

The effect of 2 dietary inclusion doses of dried Glycyrrhiza glabra leaves on the productive performance of broiler chicken was investigated.

Glycyrrhiza glabra leaves came from cultivation. Cultivation and drying followed Good Agricultural and Collecting practices. The Glycyrrhiza glabra leaves were grinded to a particle size of <0.3 mm. Diets, fed in mash form, were provided by age of bird: Starter feed fed from 0 day to 14 days, Grower feed fed from 15 days to 28 days and Finisher feed fed from 29 days to 42 days. The basal diets compositions are set forth in Table 4.

TABLE 4
Compositions of the basal starter feed,
grower feed, and finisher feed.

| | Starter | Grower | Finisher |
|---|---|---|---|
| Ingredients, in kg/100 kg | feed | feed | feed |
| Corn | 58.44 | 60.36 | 64.56 |
| Soybean meal | 30.469 | 23.82 | 19.87 |
| distiller's dried grains with solubles | 4.00 | 8.00 | 8.00 |
| meat and bone meal | 4.00 | 4.00 | 3.821 |
| soybean oil | 0.972 | 2.024 | 2.256 |
| Limestone | 0.309 | 0.287 | 0.221 |
| dicalcium phosphate | 0.609 | 0.281 | 0.110 |
| Salt | 0.391 | 0.375 | 0.379 |
| Choline | 0.064 | 0.069 | 0.069 |
| Lysine | 0.218 | 0.273 | 0.259 |
| Methionine | 0.287 | 0.266 | 0.236 |
| Threonine | 0.107 | 0.106 | 0.085 |
| Mixture supplying other minerals and | 0.143 | 0.143 | 0.143 |
| vitamins | | | |

The experimental treatments were made as follow. The Control treatment birds received basal Starter feed, basal Grower feed and basal Finisher feed. Treatment 1 birds received the basal diets supplemented with 39 g per metric ton of feed of dried ground Glycyrrhiza glabra leaves. Treatment 2 birds received the basal diets supplemented with 78 g per metric ton of feed of dried ground Glycyrrhiza glabra leaves.

Each experimental treatment was assigned to 12 pens per treatment. Each pen (dimensions of each pen are 4′×5′) contained 1 water fountain and a feed tube. Birds started on new wood shavings. 34 Ross 308 day 0 chicks were allocated to each pen. Birds were equally distributed by body weight among the 108 study pens. Continuous lighting was provided Od to 42 days.

All broilers were weighed by pen when bird ages were 0, 14, 28 and 42d. Feed was weighed in as needed and weighed out when birds were weighed. The following data were collected during the study: broiler body weights at day 0 and day 42 (to calculate body weight gain), feed intakes at day 42, feed conversion ratio and mortality at day 42. Based on these outcomes, feed conversion and adjusted feed conversion were calculated.

Feed conversion is calculated for a pen as the total feed consumed divided by (sum of total of all body weights for the pen+sum total of all mortality weight for the pen).

Adjusted feed conversion ratio with final BW adjustment at day 42 is calculated as follows: Feed conversion+(2.22 kg-actual body weight)/7.

TABLE 5
Performance of broiler chicken

| Productive | Treatments | | |
|---|---|---|---|
| response | | Treatment | Treatment |
| (unit) | Control | 1 | 2 |
| Feed intake | 3.763 | 3.769 | 3.792 |
| (kg) | | | |
| Body weight | 2.334^{A} | 2.409^{B} | 2.411^{B} |
| gain (kg) | | | |
| FCR¹ | 1.548^{A} | 1.505^{B} | 1.495^{B} |
| Adjusted | 1.565^{A} | 1.501^{B} | 1.489^{B} |
| FCR | | | |

¹FCR = Feed Conversion Ratio

The results presented in Table 5 show that the addition of Glycyrrhiza glabra ground leaves in the diet of broiler chicken improved productive performance. The addition of Glycyrrhiza glabra ground leaves increased final body weight (P=0.053) with 3.2% for both inclusion levels of Glycyrrhiza glabra leaves. Feed conversion ratio was decreased by the addition of Glycyrrhiza glabra leaves. For example, adjusted feed conversion ratio was decreased (P=0.005) with 4.1% with 39 ppm of Glycyrrhiza glabra leaves and with 5% with 78 ppm of Glycyrrhiza glabra leaves. Mortality was the same for all treatments.

### Example 4

In this experiment the effect of the dietary inclusion of dried Glycyrrhiza glabra leaves on the response of shrimps to a disease challenge was investigated. The disease challenge was an acute hepatopancreatic necrosis disease (AHPND) challenge produced by the bacteria Vibrio parahaemolyticus.

Glycyrrhiza glabra leaves came from cultivation. Cultivation and drying followed Good Agricultural and Collecting practices. The Glycyrrhiza glabra leaves were grinded to a particle size of <0.3 mm. The composition of the basal diet is shown in Table 6 below.

TABLE 6
Basal diet composition of extruded shrimp feed.

| | Inclusion levels |
|---|---|
| Ingredients | (in kg/100 kg) |
| Soya products/by-product | 35-40 |
| Wheat products/by-product | 35-40 |
| Fish meal/by-product | 8-15 |
| Macro mineral | 4-6 |
| Soy Lecithin/oil | 1.5-2.5 |
| Fish oil NA | 1.5-2 |
| Crustacean-based attractant | 1.5-3 |
| Hydrolysates | 1.5-3 |
| Amino acids mix | 0.5-1.2 |
| Vitamin-mineral premix | 0.3-0.5 |

The diets were produced as 1.4±0.1 mm extruded pellets.

The feed production procedure followed the normal guidelines for shrimp feed extrusion. First, all dried raw materials (except micro-ingredients) were ground through a 1 mm (50 Hz) grinder screen. Next, ingredients were weighed to a 0.01-g precision on an electronic scale following the formula specifications. After the extrusion, the pellets were dried until the moisture content was below 10%. Final feeds were bagged and stored at 4° C.

During the first part of the experiment, the shrimps were housed in groups and fed either the “Control” diet or the “Treatment” diet. These 2 diets were obtained as follow: the Control group received the basal diet; the Treatment group received the control diet supplemented with 1040 g of dried leaves of Glycyrrhiza glabra per metric ton of feed (1040 ppm).

The Control and the Treatment groups were each assigned to 1 plastic tank. Each tank of a capacity of 290 L was equipped with an individual filtration system and filled with artificial seawater at a salinity of 20 g/L. 75 shrimps were introduced in each tank. Shrimps were Penaeus vannamei postlarvae.

After 18 days of growth, the shrimps from these 2 groups were transferred to the disease challenge facility and housed in the infection unit (1 shrimp per tank) for acclimatization. Animals were housed individually in 10 L tanks and fed with the same diet they were fed during the first 18 days. Tanks were filled with artificial seawater with salinity of 20 g/L and equipped with mechanical/biological filter that ensured water quality. Water temperature was kept at 27° C. and the room in which the tanks were kept was illuminated 12 h a day. They were allowed to acclimatize for 3 days.

Each treatment (Control or Treatment) replicates correspond to 3 blocks of 10 individual shrimp per treatment. Shrimps were fed manually 4 times a day (9 am, 11:30 am, 2 μm, 4:30 μm) and additional feeding was provided at 8 μm and 12 μm on the days of AHPND inoculation. Shrimps were monitored twice daily for clinical signs of disease and mortality.

The challenge was an acute hepatopancreatic necrosis disease (AHPND) challenge. The bacterium used was a Vibrio parahaemolyticus isolated from shrimp suffering from AHPND. A stock of this bacterium is permanently kept frozen at −70° C. After thawing, the stock was aseptically inoculated in culture medium and grown using standard conditions. The optical density of the resulting bacterial suspension was determined spectrophotometrically. These data were used to determine the concentration of bacteria in the suspension in colony forming units per millimeter. Quantified suspensions of the bacteria were used to inoculate shrimp by immersion in all phases of this experiment. Each tank was inoculated with the same dose of bacteria.

Mortality rate was measured and cumulative mortality at 14 days post infection in (%) was calculated. The results are shown in Table 7 below.

TABLE 7
Mortality of shrimp subjected to Vibrio parahaemolyticus infection.

| | Cumulative mortality at |
|---|---|
| | 14 days post infection |
| | (%) |
| Control | 59.5% |
| Treatment | 50% |
| Effect of Treatment vs Control (%) | −16% |

The addition of Glycyrrhiza glabra leaves to the diet of shrimps significantly reduced mortality due to Vibrio parahaemolyticus.

### Example 5

The effect of dried Glycyrrhiza glabra leaves on the productive performance of broiler chicken was investigated.

Glycyrrhiza glabra leaves came from cultivation. Cultivation and drying followed Good Agricultural and Collecting practices. The Glycyrrhiza glabra leaves were grinded to a particle size of <0.3 mm. Diets, fed in mash form, were provided by age of bird: Starter feed fed from 0 day to 14 days, Grower feed fed from 15 days to 28 days and Finisher feed fed from 29 days to 42 days. The basal diets compositions were as set forth in Table 8 below.

TABLE 8
Compositions of the basal starter feed,
grower feed, and finisher feed.

| | Starter | Grower | Finisher |
|---|---|---|---|
| Ingredients, in kg/100 kg | feed | feed | feed |
| Corn | 57.02 | 58.49 | 58.189 |
| Soybean meal | 29.693 | 23.505 | 18.654 |
| distiller's dried grains with solubles | 6.00 | 10.00 | 15.000 |
| meat and bone meal | 4.00 | 4.00 | 4.000 |
| soybean oil | 1.105 | 2.213 | 2.833 |
| Limestone | 0.333 | 0.309 | 0.212 |
| dicalcium phosphate | 0.567 | 0.236 | |
| Salt | 0.433 | 0.391 | 0.342 |
| Choline | 0.062 | 0.064 | 0.054 |
| Lysine | 0.238 | 0.277 | 0.275 |
| Methionine | 0.285 | 0.260 | 0.213 |
| Threonine | 0.110 | 0.103 | 0.074 |
| Mixture supplying other minerals and | 0.154 | 0.152 | 0.154 |
| vitamins | | | |

The experimental treatments were made as follow. The Control treatment birds received basal Starter feed, basal Grower feed and basal Finisher feed. The “Treatment” was created by supplementing the basal diets with 55 g per metric ton of feed of dried and ground Glycyrrhiza glabra leaves.

Each experimental treatment was assigned to 12 pens per treatment. Each pen (dimensions of each pen are 4′×5′) contained 1 water fountain and a feed tube. Birds started on new wood shavings. 34 Ross 308 day 0 chicks were allocated to each pen. Birds were equally distributed by body weight among the 24 study pens. Continuous lighting was provided from Od to 42 days.

All broilers were weighed by pen when bird ages were 0, 14, 28 and 42d. Feed was weighed in as needed and weighed out when birds were weighed. The following data were collected during the study: broiler body weights at day 0 and day 42 (to calculate body weight gain), feed intakes at day 42, feed conversion ratio and mortality at day 42. Based on these outcomes, feed conversion and adjusted feed conversion were calculated.

Feed conversion is calculated for a pen as the total feed consumed divided by (sum of total of all body weights for the pen+sum total of all mortality weight for the pen).

Adjusted feed conversion ratio with final BW adjustment at day 42 is calculated as follows: Feed conversion+(2.22 kg−actual body weight)/7.

TABLE 9
Performance of broiler chickens at 42 days.

| Productive outcome | Control | Treatment | Effect, % |
|---|---|---|---|
| Feed intake (kg) | 4.11 | 4.16 | +1.2% |
| Body weight gain (kg) | 2.37 | 2.44 | +2.9% |
| Adjusted FCR | 1.69 | 1.65 | −2.4% |

The results presented in Table 9 show that the addition of Glycyrrhiza glabra leaves in the diet of broiler chicken improved productive performance: it increased final body weight with 2.9%. Feed conversion ratio was decreased by the addition of Glycyrrhiza glabra leaves with 2.4%.

### Example 6

The effect of dried Glycyrrhiza glabra leaves on the productive performance of salmons was investigated.

Glycyrrhiza glabra leaves came from cultivation. Cultivation and drying followed Good Agricultural and Collecting practices. The Glycyrrhiza glabra leaves were grinded to a particle size of <0.3 mm. A basal diet was formulated to meet the nutritional requirements of Atlantic salmon at ca. 41% crude protein, 28% crude lipid, 7% moisture, 24 kj/g Energy and 5% ash levels.

The experimental treatments were made as follows. The Control treatment salmons received the basal diet. The Treatment salmons received the basal diet supplemented with 130 g per metric ton of feed of dried ground Glycyrrhiza glabra leaves in place of wheat. All macro and micro ingredients, including dried ground Glycyrrhiza glabra leaves ingredients were dry mixed before the complete mix was extruded to create a 4 mm pellet.

Atlantic salmon of an average weight of 420 g were involved in the experiment with 2 treatments (Control and Treatment) in triplicate of 1 meter tanks with 40 fish per tank. The water temperature was maintained at 12° C. with a salinity of 31 ppt. Feed was provided to the salmons 3 times per day. Light regime was 24 hours with light. The experiment lasted for 54 days. Salmons were weighted on the first day (and provided mean initial weight, expressed in g), as well as on the last day of the experiment (which provided mean final weight, expressed in g). The number of feeding days was 54. Other outcomes were measured: the final biomass (expressed in g) and the total weight of dead fish (expressed in g), and the total feed eaten (expressed in g). Specific growth rate (SGR) and feed intake, both expressed in % per day were calculated as follows.

$$SGR = {( Mean final weight / Mean initial weight )}^{{( 1 / Number of feeding days )}^{- 1}} \times 100$$

$$Feed intake = SGR \times Total feed eaten / ( Final biomass - mean initial weight + Total weight dead fish )$$

Table 10 shows the result of the experiment.

TABLE 10
Performance of salmon fed Glycyrrhiza glabri leaves

| | | | Effect |
|---|---|---|---|
| | Control | Treatment | (%) |
| Feed intake, %/d | 0.527 | 0.566 | 7.4% |
| RGR¹ | 0.906 | 0.934 | 3.1% |
| SGR | 1.169 | 1.206 | 3.2% |

¹RGR is relative growth rate.

The inclusion of 130 ppm of Glycyrrhiza glabra leaves increased feed intake by 7.4% and improved specific growth rate by 3.2%.

### Example 7

In this example, the effect of Glycyrrhiza glabra leaves on mitigating the impact of Aflatoxin B1 on liver cells was studied.

Alpha mouse liver 12 cells (AML12) were grown and differentiated in DMEM/F12 medium supplemented with 10% FBS, 1% insulin-transferrin-selenium, 100 UI/mL penicillin, 100 μg/mL streptomycin, and 40 ng/ml dexamethasone at 37° C. in a humidified atmosphere with 5% CO2. The cell line was maintained by serial passages.

0.1 grams of dried Glycyrrhiza glabra leaves were suspended in 500 μL DMSO, DMSO aqueous solution (DMSO: H2O=1:1) or sterilized water to obtain stock solution. After vigorous stirring, completely dissolved the suspensions were filtered using 0.22 μm filter membrane and stored at −20° C. Purified Aflatoxin B1 was dissolved in basic medium or DMSO stored at −20° C. before dilution in cell culture media. Control samples were treated with medium.

The cytotoxic effect of Aflatoxin B1 and various concentrations of PE solution on the AML-12 cells was evaluated using the cell counting kit-8 (CCK-8) reagent. In brief, after treatment with 8 mg/L of Aflatoxin B1 with or without Glycyrrhiza glabra leave extract solutions (GG; at 10 μg/mL, 15 μg/mL and 20 μg/mL) for 24 h, 10 μL CCK-8 solution was added according to the instructions provided by the manufacturer. A control was used for which no Aflatoxin B1 and no At the indicated time, the absorbance at 450 nm was determined by a microplate reader. The results are shown in Table 11 below.

TABLE 11
Cell viability (%) of

| | Cell viability |
|---|---|
| Treatment | (%) |
| [1] Control | 100 |
| [2] = [1] + 8 mg/L of Aflatoxin B1 | 51.8 |
| [3] = [2] + 10 ug/mL of GG | 55.9 |
| [4] = [2] + 15 ug/mL of GG | 64.7 |
| [5] = [2] + 20 ug/mL of GG | 74.0 |

It can be concluded that Glycyrrhiza glabra leaves mitigate the effects of Aflatoxin B1 on cells.

### Example 8

Newly hatched chickens from Longnecker's hatchery, Elizabethtown, PA were housed in a Petersime brooder units and provided with feed and water ad libitum. All chickens were weighed and allocated to a cage. Per treatment, 60 chickens (5 birds/cage=12 replication) were used. Initial body weight was the same among treatments.

For E. maxima infection freshly propagated E. maxima (ARS strain) were used to infect the birds. On d 14 after hatch, the birds were infected with 10,000 E. maxima oocysts through oral gavage.

The experimental treatments were the following: [1]=Negative control (NC), non infected and untreated chicken; [2]=Positive control (PC)=[1]+infection; [3]=[2]+90 mg/kg feed of monensin (MO); [4]=[2]+55 ppm of dried Glycyrrhiza glabra leaves; [5]=[2]+160 ppm of dried Glycyrrhiza glabra leaves. Monensin is the current standard used to control coccidiosis.

Birds were weighed individually on d 7, 14, 18, 20, and 23 to calculate the weight gain.

Oocyst shedding: Faeces from each group was collected separately from 6-8 days post infection (dpi) for coccidiosis. To count coccidia oocysts, various dilutions were made initially to determine the optimum dilutions for enumeration of oocysts for each sample. Oocysts were counted microscopically using a McMaster counting chamber using a sodium chloride flotation method. The total number of oocysts shed per chicken were calculated using the formula: total oocysts/bird=(oocyst count×dilution factor×fecal sample volume/counting chamber volume)/number of birds per cage.

### Results

The performance results are shown in Table 12 below.

TABLE 12
Performance data of chickens without (NC) or with (PC,
MO, GG) E. maxima infection in the absence of further
treatment (PC) or with treatment using monensin (MO), 55
ppm Glycyrrhiza glabra leaves (55 ppm GG) or 160 ppm
Glycyrrhiza glabra leaves (160 ppm GG).

| | | | | 55 | 160 |
|---|---|---|---|---|---|
| | | | | ppm | ppm |
| | NC | PC | MO | GG | GG |
| BW, g | | | | | |
| d 19 (4 dpi) | 853 | 803 | 890 | 853 | 825 |
| d 21 (6 dpi) | 1,007 | 748 | 1,023 | 881 | 859 |
| d 23 (8 dpi) | 1,170 | 763 | 1,220 | 956 | 919 |
| ADG, g | | | | | |
| d 15 to 19 | 69.3 | 59.2 | 76.3 | 68.8 | 63.9 |
| (0 to 4 dpi) | | | | | |
| d 15 to 21 | 71.8 | 30.3 | 72.9 | 50.5 | 48.2 |
| (0 to 6 dpi) | | | | | |
| d 15 to 23 | 74.3 | 24.6 | 79.3 | 46.4 | 43.7 |
| (0 to 8 dpi) | | | | | |
| ADFI, g | | | | | |
| d 15 to 19 | 90.9 | 83.4 | 90 | 84.2 | 82.8 |
| (0 to 4 dpi) | | | | | |
| d 15 to 21 | 94.5 | 75.6 | 93.5 | 84.8 | 80.2 |
| (0 to 6 dpi) | | | | | |
| d 15 to 23 | 95.4 | 70.7 | 94.3 | 79.2 | 75.3 |
| (0 to 8 dpi) | | | | | |
| FCR | | | | | |
| d 15 to 19 | 1.32 | 1.49 | 1.191 | 1.23 | 1.33 |
| (0 to 4 dpi) | | | | | |
| d 15 to 21 | 1.32 | 3.35 | 1.31 | 1.76 | 1.68 |
| (0 to 6 dpi) | | | | | |
| d 15 to 23 | 1.29 | 2.19 | 1.21 | 1.92 | 1.88 |
| (0 to 8 dpi) | | | | | |

dpi means days post-infection; BW means bodyweight; ADG means average daily gain; ADFI means average daily feed intake; FCR means feed conversion ratio.

Monensin showed excellent effects in preserving growth performance in chickens infected with E. maxima. It was found that dietary supplementation with dried Glycyrrhiza glabra leaves also improved the growth performance in chickens infected with E. maxima.

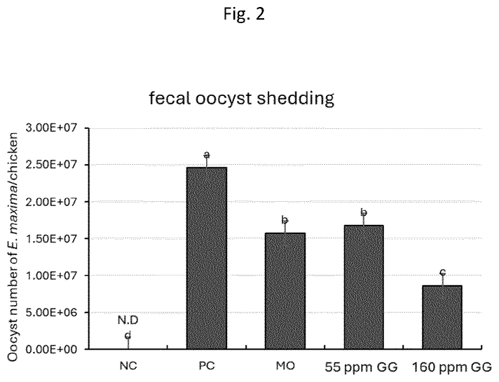

FIG. 2 shows the results for fecal oocyst shedding. It was found that monensin reduced the lesion score and the number of oocysts, corresponding to the growth results. Glycyrrhiza glabra leaves also significantly reduced the number of oocysts. At 160 ppm addition of Glycyrrhiza glabra leaves to feed, the oocyst number was even lower than that of monensin.

### Example 9

The effect of the dietary inclusion of Glycyrrhiza glabra leaves on the response of shrimps to a viral disease (white spot disease or white spot syndrome) was investigated.

Glycyrrhiza glabra leaves came from cultivation. Cultivation and drying followed Good Agricultural and Collecting practices. The Glycyrrhiza glabra leaves were grinded to a particle size of <0.3 mm. The basal diet composition was as set forth in Table 13.

TABLE 13
Basal diet composition of extruded shrimp feed.

| | Inclusion levels |
|---|---|
| Ingredients | (in kg/100 kg) |
| Soya products/by-product | 35-40 |
| Wheat products/by-product | 35-40 |
| Fish meal/by-product | 8-15 |
| Macro mineral | 4-6 |
| Soy Lecithin/oil | 1.5-2.5 |
| Fish oil NA | 1.5-2 |
| Crustacean-based attractant | 1.5-3 |
| Hydrolysates | 1.5-3 |
| Amino acids mix | 0.5-1.2 |
| Vitamin-mineral premix | 0.3-0.5 |

The diets were produced as 1.4±0.1 mm extruded pellets.

The feed production procedure followed the normal guidelines for shrimp feed extrusion. After the extrusion, the pellets were dried until the moisture content was below 10%. Final feeds were bagged and stored at 4° C.

The experimental treatments were made as follow. The ‘Control’ shrimps received basal shrimp feed. ‘Treatment 1’ shrimps received the basal diet supplemented with 1040 g per metric ton of feed (1040 ppm) of dried and ground Glycyrrhiza glabra leaves. ‘Treatment 2’ shrimps received the basal diet supplemented with 1040 g per metric ton of feed (1040 ppm) of dried and ground Glycyrrhiza glabra leaves and 150 g per metric ton of capsicum oleoresin.

The shrimps were housed individual in 10 L glass tanks filled with artificial seawater composed by adding a commercial salt mixture to purified water at 20 g/L. The tanks were equipped with individual mechanical/biological filter. No water was shared between the tanks. Water quality was maintained by filtration, aeration and regular water changes. Water temperature was kept at 27° C. The room was illuminated 12 h a day. Shrimps were fed 3 times a day. The shrimps were monitored twice daily for clinical signs of disease and mortality.

The treatment replicates corresponded to 3 blocks of 10 individual shrimps per treatment. Blocks were placed at different location of the challenge setup.

Three days before starting the disease challenge, shrimps were transferred to the disease challenge setup. Shrimps were fed with different treatment diets. A white spot syndrome virus (WSSV) was used in this experiment to confer the disease challenge. A virus stock was kept frozen at −70° C. at the facilities. A solid WSSV inoculum was created and used in the oral infection experiment. The evaluated parameter was mortality (%). The results are shown in Table 14 below.

TABLE 14

| | | Effect compared |
|---|---|---|
| Treatment | Final mortality (%) | to control, % |
| Control | 46.7 | — |
| Treatment 1 | 40.0 | −14% |
| Treatment 2 | 33.3 | −29% |

The addition of Glycyrrhiza glabra leaves improved survival in response to the WSSV virus infection in shrimp. The addition of a capsicum oleoresin containing products further improved the resistance to the virus.

### Example 10

This experiment aimed at testing efficacy of dried Glycyrrhiza glabra leaves in transition dairy cows, with a first dose tested of 260 mg/head/day.

30 multiparous (parity 2+) dairy cows blocked by BW, previous MY, and expected calving, and assigned to one of the following treatments for weeks-3 to +6 of lactation (n=15/trt). The treatments were the following: 1) control; 2) 260 mg/head/day dried Glycyrrhiza glabra leaves.

Measurements taken throughout the study: i) Individual feed intake (daily); Individual milk yield (daily); Milk composition (weekly; fat, protein, lactose); Individual body weight (weekly).

Daily data were subjected to 2-way ANOVA. Weekly data were submitted to 1-way ANOVA with repeated measurements.

### Results and Conclusion

The results are shown in Table 15 below.

TABLE 15
Performance parameters of dairy cows.

| | Treatment | | |
|---|---|---|---|
| | CON | GG | P-values |
| DMI, kg | 19.0 | 19.1 | 0.95 |
| Milk yield, kg/d | 41.4 | 40.8 | 0.87 |
| BW, kg | 617 | 637 | 0.05 |
| Milk fat, % | 4.27 | 4.48 | 0.05 |
| Milk protein, % | 3.28 | 3.31 | 0.80 |

It was found that supplementation of dried Glycyrrhiza glabra leaves to feed of dairy cows in the transition period increased the percentage of milk fat in the milk (p=0.05) and increased bodyweight of the cows (p=0.05).

### Example 11

### Objective

The overall goal of the project was to determine the efficacy of a composition comprising cinnamon oil, tea extract, and pomegranate extract (CO/TE/PE; 1:1:1) in ameliorating parasite load and shedding, disease development and pathogenesis, and augmentation of animal host protective innate mucosal immunity during Cryptosporidium parvum infection using a transgenic mouse infection model.

### Materials and Methods

Twenty-four (24) 8-week-old male interferon-gamma knockout (IFN-γ KO) mice (B6.129S7-IfngTM1 Ts/J) were purchased and allowed to quarantine and acclimatize for 1 week before the commencement of experiments. There were three treatment groups, with 8 individually housed mice per treatment. The treatments consisted of: Group 1, uninfected-untreated; Group 2, C. parvum-infected-untreated (Control, reconstitution reagent DMSO only); Group 3, infected-dried Glycyrrhiza glabra leaves. Each mouse from the infected groups was infected by oral gavage administration of 104 C. parvum AUCP-1 isolate oocysts on Day 3 after the experiment started.

### 1. Parasites

The Cryptosporidium parvum AUCP-1 isolate was maintained and propagated in male Holstein calves. C. parvum oocysts were extracted and purified from freshly collected calf feces by sequential sieve filtration, Sheather's sugar flotation, and discontinuous sucrose density gradient centrifugation. Purified oocysts were washed and stored in phosphate-buffered saline (PBS) at 4° C. and used within 3 months to ensure maximum viability as judged by excystation.

### 2. Animals

Male IFN-γ knockout mice (B6.129S7-Ifngtm1 Ts/J), aged 7 weeks, were procured from The Jackson Laboratory, USA, and housed under Biosafety level 2 conditions. Feed (commercial mouse chow) and water were supplied ad libitum. Animals were left to acclimatize for 4 days before the start of experiments.

3. Glycyrrhiza glabra Preparation

Dried Glycyrrhiza glabra leaves in powder form was reconstituted in molecular grade dimethyl sulfoxide (DMSO) followed by filter-sterilization using a 0.22μ filter, and stored at room temperature in the dark until use.

### 4. Treatment, Infection, Sample Collection and Determination of Physical Parameters

Mice were divided into three groups with 8 mice per group. Within each group, each individual mouse was housed in an individual cage. The groups were as follows:
Uninfected untreated group (UU).
Infected DMSO-treated group (DMSO).
Infected GG-treated group

Three days prior to infection with C. parvum, mice in the various treatment groups begun receiving daily oral gavage administration of compounds or DMSO as follows: GG=55 mg/kg; DMSO=0.05 ml DMSO; UU=0.05 ml DMSO. On the third day after commencement of treatment, mice in the infected groups (DMSO, GG) each received 104 C. parvum oocysts suspension in PBS by oral gavage, and the respective daily treatments per group continued for 14 days post-infection. Alongside treatment, mice were provided regular commercial chow diet and water ad libitum. During the entire period of treatment, fecal samples were collected daily, and physical fecal consistence was determined. Animals were also scored for physical state and activity, body weight and appetite. Moreover, mice were sacrificed and their distal small intestines used for histopathological examination.

### Results

### 1. Physical Parameters:

Generally, the Uninfected Untreated (UU) mice were active, healthy, with good appetite throughout and gained weight. The weight gain and activity observed is typical of healthy mice at this age (7 weeks), as they would still be in growth phase. The Infected DMSO-treated (DMSO) depicted typical cryptosporidiosis disease progression, with diarrhea, weight loss, poor body condition and reduced activity. Overall, they lost weight over the experimental period despite that that they were still growing.

The infected-GG-treated mice had better physical outcomes than the DMSO-group. They did not develop diarrhea, they remained active and healthy looking throughout, and they gained weight, though not as much as the uninfected mice did. At post-mortem, they had healthy-looking intestines, without hemorrhages. From these observations, it was evident that treatment with GG ameliorated cryptosporidiosis and improved the health of the infected animals.

2. Effect of Compound Treatment on C. parvum Oocysts Load in Infected Mice Feces:

As expected, the Infected DMSO-treated control group mice showed a progressive increase in oocysts shedding, with the first a peak load of about 2×108 oocysts per gram of feces (OPG) being attained by day 10 post-infection, with a second wave higher peak of 2.2×108 oocysts being observed at 15 days post-infection. Treatment with GG depicted oocysts loads that were consistently significantly (P<0.05) lower than those of the infected DMSO-treated mice oocysts throughout the treatment period. These results corroborated the physical parameters that indicated that GG treatment ameliorated disease progression and outcome.

### 3. Amelioration of Histopathological Lesions:

At the end of the treatments, mice were sacrificed and their distal small intestines used for histopathological examination. As expected, the uninfected control group mice showed healthy intestinal mucosa with prominent villi. On the other hand, the infected DMSO-treated mice had intestinal lesions characterized by mucosal erosion, villous atrophy, hypertrophy of the crypts, and accumulation of inflammatory cells. Mice treated with GG showed better outcomes than the infected DMSO-treated mice, depicting mostly intact mucosa with prominent villi. While GG-treated mice showed accumulation of inflammatory cells in the mucosa, there was no evident hypertrophy of intestinal crypts, indicating that these mice still had the ability to replenish their intestinal epithelial cells from the intact crypts. These findings are consistent with the significantly reduced number of oocysts detected in GG-treated mice, and thus indicate that this treatment ameliorated parasite proliferation and intestinal pathology in the infected mice.

### Example 12

The goal of this experiment was to determine the efficacy of Glycyrrhiza glabra leaves in ameliorating parasite load and shedding, disease development and pathogenesis, and augmentation of animal host protective innate mucosal immunity during Cryptosporidium parvum infection using a bovine calf infection model.

### Experimental Approach

### Determination of Efficacy in Ameliorating Cryptosporidiosis

GG (Glycyrrhiza glabra leaves, dried and ground into powder form, reconstituted in molecular grade dimethyl sulfoxide (DMSO) and stored at room temperature in the dark while in use) was individually administered to calves by oral gavage once daily for 3 days prior to infection, and continued for 14 days post-infection with C. parvum. Calves were further provided a milk-replacer diet and water ad libitum. During the entire period of treatment, fecal samples were collected daily for measurement of C. parvum oocysts shedding by quantitative real-time PCR, and physical fecal consistence determination. Animals were also scored for physical state and activity, body weight, feeding, body temperature, fur condition and body posture.

The experiments consisted of 3 groups with 5 calves per group as follows:
5 C. parvum-infected calves treated with GG (13.75 g/kg BW)
5 C. parvum-infected calves, untreated.
5 uninfected, untreated calves

### Results and Conclusion

Generally, the Uninfected Untreated calves were active, healthy, with good appetite throughout and gained body weight. The weight gain and activity observed is typical of healthy calves at this age, as they would still be in rapid growth phase. The Infected DMSO-treated (DMSO) calves depicted typical cryptosporidiosis disease progression, with diarrhea, weight loss, poor body condition and reduced activity. They progressively became sicker with time and two calves died of illness at 10 days and 12 days post-infection, respectively. Overall, all calves in this group lost weight over the experimental period despite being in rapid growth age range. The infected-GG-treated calves had better physical outcomes than the infected untreated group. Initially, 2 animals in this group developed diarrhea at 3 days post-infection but they recovered within 2-3 days, though one calf remained a bit lethargic for several days. The rest of the calves in GG group were active throughout, and they gained weight, albeit not as much as the uninfected calves did. From these observations, it was evident that treatment with GG ameliorated cryptosporidiosis and improved the health of the infected animals.

As expected, the Infected DMSO-treated control group calves showed a progressive increase in oocysts shedding, with the peak load of about 2.5×10⁸ oocysts per gram of feces (OPG) being attained by day 9 post-infection. Treatment with GG depicted oocysts loads that were consistently significantly (P<0.05) lower than those of the infected DMSO-treated calves throughout the treatment period, with the first peak of 6.5×10⁴ oocysts per gram feces being observed at 8 days post-infection, while the second wave of oocysts shedding had a peak of 5.5×10⁴ oocysts per gram feces at day 11 post-infection. These results corroborated the physical parameters that indicated that GG treatment ameliorated disease progression and outcome.

At the end of the treatments, calves were sacrificed and their distal small intestines used for histopathological examination. As expected, the uninfected control group calves showed healthy intestinal mucosa with prominent villi. On the other hand, the infected DMSO-treated calves had intestinal lesions characterized by villous atrophy. Notably, calves treated with GG showed better outcomes than the infected DMSO-treated calves, depicting mostly intact mucosa with prominent villi. On average, the uninfected, infected-DMSO-treated, and infected-GG-treated calves had villi that measured 300, 260 and 285 μm, respectively. These findings are consistent with the significantly reduced number of oocysts detected in GG-treated calves, and thus indicate that the GG treatment ameliorated parasite proliferation and intestinal pathology in the infected calves.

### Example 13

The objective of this experiment was to see whether treatment with dried Glycyrrhiza glabra would have a beneficial effect on parasite infection with Enterocytozoon hepatopenaei (EHP), one of the most prevalent pathogens and poses a major challenge in shrimp aquaculture worldwide. It is an intracellular parasitic disease that is associated with stunted growth in farmed shrimp, high size variation, and numerous economic losses for shrimp farmers.

### Materials and Methods

The trial design consisted of 3 groups: a negative control, a positive control, and treatment with dried Glycyrrhiza glabra leaves (“GG Treatment”). The trial was set up as a completely randomized design (CRD) with 50 shrimp per tank. The trial was carried out for 63 days: including 14 days for pre-challenge, 7 days for EHP cohabitation challenge, and a following post-challenge period for 42 days.

Shrimp were fed with their respective diet at satiation for 4 meals per day during the trial. Shrimp were fed at 5% BW in the pre-challenge, 3% BW during EHP infection challenge and 5% BW in the post-challenge. Amount of feed was adjusted depending on the estimated biomass and shrimp feeding behaviour of the tanks.

EHP-infected shrimp (stock inoculums) having an average EHP load/density of 108-109 copies per gram EHP-infected shrimp (quantified using qPCR) were used for the challenge. The tanks of the GG treatment and the positive control were challenged by the co-habitation method with 10 EHP-infected shrimp for each tank. Negative control tanks were co-cultured with 10 specific pathogen-free (SPF) shrimp. The challenge lasted for 7 days of cohabitation. Then, the inoculum (EHP-infected shrimp) and SPF shrimp were removed from the tanks.

The SPF and infected shrimp were separated by a net divider that allowed for the free exchange of water. The co-habitation challenge method is designed as follows: in each 350 L plastic tank, a 40 cubic litter rectangular net is fitted. During the challenge, 10 EHP-infected shrimp were stocked inside the suspended net, and the experimental shrimp were stocked outside at the same time. Furthermore, 20 L of water from the EHP-infected inoculum tank was supplied into the Positive control and the GG treatment tanks to facilitate the challenge.

Good water quality was maintained during the study for optimal growth and survival of shrimp over the trial duration.

### Results

Survival rates of shrimps are shown in Table 16 below.

TABLE 16
Survival rates of shrimp at the trial termination

| Treatment | Survival rate (%) |
|---|---|
| Negative control | 90.00 ± 0.00 |
| Positive control | 49.00 ± 1.15 |
| GG Treatment | 66.50 ± 3.00 |

The highest survival of shrimp was found with GG treatment, which was significantly higher than the positive control (P<0.05).

At the trial termination, shrimp were harvested and weighed for calculating growth performance indexes. The growth performance parameters of shrimp at the termination day are described in Table 17.

TABLE 17
Growth performance parameters after 63 days of culture.

| | Initial | Final | Mean | | | | |
|---|---|---|---|---|---|---|---|
| | mean | mean | weight | | | Feed | |
| | weight | weight | gain | ADG | SGR | consumption | |
| Treatment | (g) | (g) | (g) | (g/day) | (%/day) | (g) | FCR |
| Negative | 1.10 | 12.51 | 11.41 | 0.20 | 3.86 | 594.48 | 1.17 |
| control | | | | | | | |
| Positive | 1.10 | 5.43 | 4.33 | 0.09 | 2.53 | 288.55 | 3.97 |
| control | | | | | | | |
| GG | 1.10 | 5.70 | 4.60 | 0.09 | 2.61 | 286.80 | 2.33 |
| Treatment | | | | | | | |

Infection with EHP clearly affected the performance of the shrimp (“Positive control”). Final mean body weight, mean body weight gain, average daily gain, specific growth rate (SGR), Feed consumption and feed conversion ratio (FCR) were all negatively affected by infection with EHP. Treatment with GG was able to improve the performance parameters relative to the Positive Control.

It was found that at the end of the trail, the Positive Control had an average EHP load of 6.6×10⁸. The GG Treatment group had an average EHP load of 4.73×10⁵, which is significantly lower than the Positive Control. Therefore, based on the EHP load as a measure of efficacy, GG treatment reduced the EHP load in the shrimp after infection.

In conclusion, treatment with Glycyrrhiza glabra leaves improved the outcome of EHP infection in terms of performance as well as mortality.

1-20. (canceled)

21. An animal feed comprising Glycyrrhiza glabra aerial parts, or an extract of Glycyrrhiza glabra aerial parts, wherein the Glycyrrhiza glabra aerial parts are included in an amount of about 1 mg/kg to about 10 g per kg of the animal feed.

22. The animal feed according to claim 21, wherein the Glycyrrhiza glabra aerial parts are dried, and optionally ground.

23. The animal feed according to claim 21, wherein the Glycyrrhiza glabra aerial parts comprise Glycyrrhiza glabra leaves.

24. The animal feed according to claim 21, wherein the extract of Glycyrrhiza glabra aerial parts is selected from an aqueous extract, ethanolic extract, methanolic extract, isopropanolic extract, ethyl acetate extract, acetonic extract, hexane extract, supercritical CO₂ extract, and combinations thereof.

25. A method of feeding an animal, comprising administering to the animal the animal feed according to claim 21.

26. A feed additive comprising Glycyrrhiza glabra aerial parts, or an extract of Glycyrrhiza glabra aerial parts.

27. The feed additive according to claim 26, wherein the Glycyrrhiza glabra aerial parts comprise Glycyrrhiza glabra leaves.

28. A method for feeding an animal, the method comprising administering to the animal the feed additive of claim 26 and an animal feed, wherein the feed additive is included in an amount of about 1 mg/kg to about 10,000 mg per kg of the animal feed.

29. The method according to claim 28, wherein the extract of Glycyrrhiza glabra aerial parts is selected from an aqueous extract, ethanolic extract, methanolic extract, isopropanolic extract, ethyl acetate extract, acetonic extract, hexane extract, supercritical CO2 extract, and combinations thereof.

30. The method according to claim 28, wherein the animal feed is for a farming animal or companion animal.

31. The method according to claim 30, wherein the farming animal is selected from poultry, swine, ruminant, fish, and crustacean.

32. The method according to claim 30, wherein the companion animal is selected from an ornamental fish, cat, dog, horse, rabbit, guinea pig, and hamster.

33. A method of increasing performance of an animal, comprising administering to the animal the animal feed according to claim 21.

34. The method according to claim 33, wherein increasing performance comprises increasing one or more or all of the animal's feed intake, average daily gain, feed efficiency, relative growth rate, specific growth rate, and milk fat yield.

35. A method of preventing, alleviating the severity of, and/or reducing microbial infection in an animal, the method comprising administering to the animal the animal feed according to claim 21.

36. The method according to claim 35, wherein the microbial infection is caused by a microbe selected from a bacterium, a virus, a fungus, a yeast, and a parasite.

37. The method according to claim 35, wherein the microbial infection is caused by a Gram-negative bacterium.

38. The method according to claim 35, wherein the microbial infection is caused by a virus.

39. The method according to claim 35, wherein the microbial infection is caused by a parasite.

40. A method of preventing, reducing the severity of, and/or treating liver toxicity or liver damage due to mycotoxin exposure in an animal, the method comprising administering to the animal the animal feed according to claim 21.
